(19) United States
(12) Patent Application Publication
(10) Pub. No.: US 2015/0351947 A1
(43) Pub. Date: Dec. 10, 2015

(54) BACK SUPPORT

(71) Applicant: V V/ANJA C SEILER HANSEN, Middelfart (DK)

(72) Inventor: Anja Camilla Seiler Hansen, Middelfart (DK)

(21) Appl. No.: 14/759,993

(22) PCT Filed: Dec. 18, 2013
(86) PCT No.: PCT/DK2013/050440

§ 371 Date: Jul. 9, 2015

(30) Foreign Application Priority Data
Jan. 9, 2013 (DK) PA 2013 70008

(51) Int. Cl. A61F 5/02

(52) U.S. Cl. CPC A61F 5/028

(57) ABSTRACT

A back support including a waist band with at least one front-facing fixing region and at least one back-facing supporting plate attached to the waist band; the back support further includes at least two first leg straps fixed in both ends unto the waist band in the fixing region; the first leg straps each forming a loop extending in use from the fixing region downwardly and behind a respective leg of the user and again upwardly to the fixing region; the at least one supporting plate being adapted to support the lower back region of the user.

### FIELD OF THE INVENTION

The present invention relates to the field of back supports.

### BACKGROUND OF THE INVENTION

There may be various reasons for a back support may be relevant. For instance it may be in relation to merely adjusting posture or for comfort reasons. In other instances it may be relevant in relation to back damages and back problems in general. In a situation where a person severely injures his back, e.g. a broken back, in order to recover it is necessary to provide a satisfying support for the back for at least a number of months to let the body's own restoring process occur unhindered.

An offer from the health care system in Denmark in this regard is a rigid back support mounted around the torso of the patient, on the back with one plate stretching from the pelvis to approximately halfway up the back, and on the front with three plates rigidly connected; a first stretching out over the thorax, a second stretching out over the abdomen and a third stretching out over the upper part of the pelvis. As this back support is rigid and stretches out over the main part of the front and back of the torso, it is really uncomfortable to wear. For instance, it impedes breathing and eating due to the constant pressure exerted on the abdomen and thorax. Furthermore, it is difficult to handle and very difficult to clean.

It is an object of the invention to provide an improved back support.

### SUMMARY OF THE INVENTION

The invention relates to a back support comprising a waist band with at least one front-facing fixing region and at least one back-facing supporting plate attached to said waist band; said back support further comprises at least two first leg straps fixed in both ends unto said waist band in said fixing region; said first leg straps each forming a loop extending in use from the fixing region downwardly and behind a respective leg of the user and again upwardly to the fixing region; said at least one supporting plate being adapted to support the lower back region of the user.

With the present invention the before-mentioned disadvantages with the prior art has been overcome. Like in the prior art, the back support according to embodiments of the present invention provides a pressure exerted against at least the lower part of the user's spine, thereby providing a desired support. However, contrary to the prior art, the back support according to embodiments of the present invention is very comfortable to wear i.a. in that pressure exerted on the thorax may be avoided and pressure on the abdomen may be substantially lessened as compared to the prior art back support. These advantages may be observed for both sitting and standing positions of the user.

In an embodiment of the invention, said back support further comprises at least two second leg straps fixed in both ends unto said waist band in said fixing region; said second leg straps each forming a loop extending in use from the fixing region downwardly and behind a respective leg of the user and again upwardly to the fixing region; wherein said at least two first leg straps are adapted for, when in use, tightening behind the upper region of the thigh of the user, and said at least two second leg straps are adapted for, when in use, tightening behind the lower region of the thigh of the user.

With two leg straps on each thigh of the user, a very satisfying embodiment is obtained. With an ideal adjustment of the length of each strap, a satisfying pressure to the back is believed to be possible.

In an embodiment of the invention, said supporting plate is substantially shaped as the shape of a back of a user to increase the comfortability for the user.

According to some embodiments, the supporting plate is shaped to touch the back over a wide area, such that a large pressure can be applied with only very little annoyance to the user.

In an embodiment of the invention, said supporting plate comprises a pad to increase the comfortability for the user.

According to some embodiments, the supporting plate may include or be formed of a relative soft material for comfort reasons. In some embodiments the supporting plate may comprise a pad formed of one or more suitable materials, e.g. a textile webbing sleeve having a filling of a suitable resilient rubber material, a gel or a silicone rubber. According to various embodiments, the supporting plate and/or an optional pad may at least comprise a material such as leather, plastic, fabric, elastic material, etc. A skilled person will be able to vary within common suitable supporting materials.

In an embodiment of the invention, said supporting plate comprises means for actively providing heating, cooling, and/or electrical impulses.

For some back patients, cooling or heating may be very pleasant and helpful. Furthermore, electrodes may provide electrical impulses, also helpful to some users.

In an embodiment of the invention, said waist band comprises a buckle for adjusting the circumference of the waist band.

The waist band should be adjustable to fit the user and furthermore to enable tightening to a desirable degree.

In an embodiment of the invention, said straps comprises buckles for adjusting the length of said straps.

The straps should be adjustable to enable tightening to a desirable degree to secure a sufficient pulling force to the back support.

In an embodiment of the invention, said back support further comprises a connecting means for connecting said waist band to a fixed structure in front of the user, preferably said connecting means comprises a hook, a snap hook and/or a strap comprising a loop.

The present invention may be ideally suitable for travelling on e.g. busses, trains, airplanes etc. Normally such travelling may be problematic and uncomfortable for people with a bad back; however, with the present invention it may be even highly comfortable to be on public transportation. When seated, the full pressure exerted on the back may then be provided by the seat in front of the user. Preferably the connecting means should comprise a length adjusting mechanism in order to fit any distance to a front seat.

In an embodiment of the invention, said back support further comprises a lower strap system for, when in use, assisting said second leg straps in maintaining their position on the lower region of the thigh of the user; said lower strap system comprising at least one strap surrounding the leg of said user.

In an embodiment of the invention, said lower strap system, when in use, comprises two straps on each leg of said user, one above the knee and one below the knee, both surrounding the legs, and at least one connecting strap for connecting said straps on each leg.

In an embodiment of the invention, said back support further comprises an upper strap system for, when in use, assisting waist band in maintaining its position around the waist; said upper strap system comprising at least one strap extending around the shoulder of the user.

Further to the already described straps, in some embodiments it may be advantageous to include straps supported over the shoulders of the user.

In an embodiment of the invention, said back support further comprises an upper strap system for, when in use, assisting waist band in maintaining its position around the waist;

In an embodiment of the invention, the straps are formed of an elastic material.

The straps may be provided in elastic materials, varying in width, elasticity, strength, flexibility and carrying capacity. Typical widths may be between 1 and 10 cm, such as between 3 and 7 cm. The elastic material may be any within the art commonly used stretchable fabric or elastomer.

In an embodiment of the invention, said at least one back-facing supporting plate has an area of more than 100 cm², preferably more than 200 cm².

The area of the supporting plate may vary in various embodiments, according to the need or desire of the user. In some embodiments the area of the supporting plate is adjustable.

In an embodiment of the invention, said at least one back-facing supporting plate is fixed on said waist band or said waist band is connected to said supporting plate in each side of said supporting plate.

Moreover the invention relates to the use of a back support according to any of the preceding claims for supporting the back of a user.

Moreover the invention relates to a method of supporting the back of a user by wearing a back support according to any of the preceding claims.

### THE FIGURES

The invention will now be described with reference to the drawings of which

FIG. 1 illustrates a back support according to an embodiment of the invention seen from the back,

FIG. 2 illustrates the same embodiment as in FIG. 1 seen from the front,

FIG. 3 illustrates a prior art back support seen from the back, and

FIG. 4 illustrates the same prior art back support seen from the front.

### DETAILED DESCRIPTION

The term “buckle” is intended to broadly cover standard tightening mechanisms such as Velcro and mechanisms commonly known from e.g. sports bags and seat belts.

The term “front-facing fixing region” is intended to cover the part of the waist band in the front used for fixing. This fixing can occur in a number of different suitable well-known ways, typically by some kind of buckles, and thus the size of the fixing region on the waist band may vary as well. However, in preferred embodiments the fixing region has a horizontal extension of less than 30 cm, such as less than 20 cm, centered on the navel of the user.

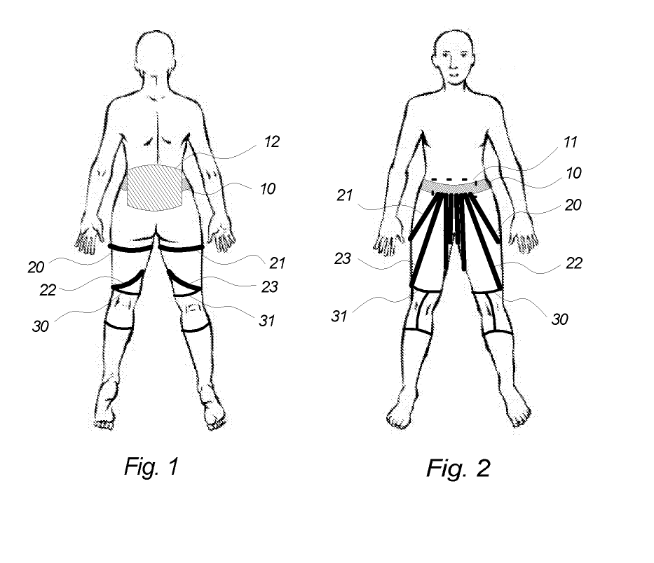

FIGS. 1 and 2 illustrate a back support according to an embodiment of the invention. The user carries a waist band 10 comprising or connected to a supporting plate 12 exerting a pressure against the back of the user. In the shown embodiment, the plate 12 may provide excellent support for e.g. a damaged lower part of the user's spine. The pressure exerted against the back may be provided to a certain degree by the direct tightening of the waist band 10 but mainly by the pulling effect from a number of straps 20, 21, 22, 23 on the front of the user. This pulling effect is created by fastening both ends of the straps 20, 21, 22, 23 onto said waist band 10 within a fixing region 11 on the front side of the waist band 10, leading the straps around the legs as shown in the figures thereby leading the legs to provide a pulling force unto the waist band resulting in a pressure exerted onto the supporting plate against the back of the user.

In the shown embodiment two first leg straps 20, 21 are positioned on the upper region of the thighs and two second leg straps 22, 23 are positioned on the lower region of the thighs. Hereby all four straps may aid in providing an increased pressure exerted against the back. However, in another embodiment the back support may only comprise the two first leg straps (20, 21) arranged in the upper region of the thighs or the back support may only comprise the two second leg straps (22, 23) arranged in the lower region of the thighs.

The upper straps 20, 21 will normally remain in position as being restricted in movement by the crotch of the user, whereas the lower straps 22, 23 advantageously may be supported further to be held in position. Such further support is in the shown embodiment provided by a lower strap system 30, 31, which may be connected in different known ways to the lower straps 22, 23 thereby aiding them in maintaining the position on the legs of the user.

In the shown embodiment the strap system comprises two straps surrounding the legs, above and below the knees respectively, connected with a further strap. Obviously, the shape of the strap system may be varied a lot by the skilled person while still providing the desired extra support for the lower straps 22, 23. In another embodiment the position of the lower second leg straps 22, 23 or even the upper first leg straps 20, 21 could be substantially fixed on or in relation to the user's legs by means of a belt, a knee bandage, adhesive tape or other.

It should be noted that the position to be fixed or maintained by the straps 20, 21, 22, 23 on the legs of the user are primarily the lowest position of the respective straps 20, 21, 22, 23—i.e. the above mentioned lower strap system 30, 31 would only fix the lower straps 22, 23 in the lowest position on the user's legs thereby leaving the rest of the length of the straps 22, 23 free to adapt to the user's current position, cloth and other.

It should also be noted that the attachments of the straps in the fixing region are shown very simplified in FIG. 2. This attachment may be carried out according to conventional attachment methods, such as snap hooks, buttons, Velcro, tying a knot etc.

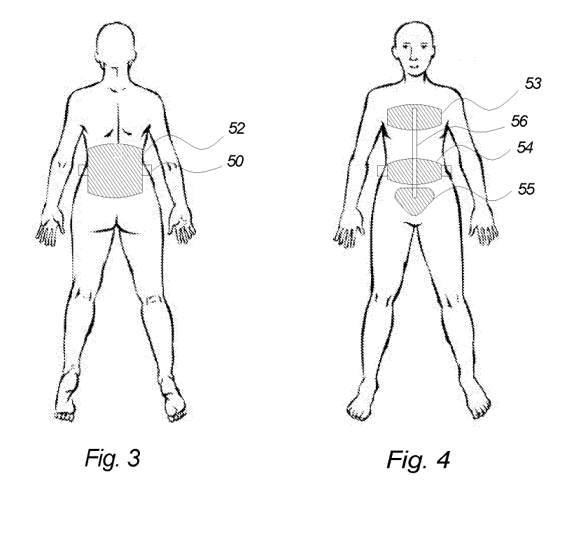

FIGS. 3 and 4 illustrate a prior art back support. On the back a supporting plate 52 supports the back similarly to in the present invention. However, in this prior art back support, the pulling effect is created by a constant pressure on the front of the user exerted by three supporting plates 53, 54, 55 on the front, all three connected by a connecting guide 56. All elements of this prior art back support is rigid, also the connection 50 between the front and the back; consequently, this prior art back support holds the torso in a very fixed position, thereby making it unpleasant to wear.

It is noted that the curvature of the lower back of human beings is believed to facilitate the invention in that the back support when placed on the lower back will typically not be vertically placed on the back but instead have a forward inclination. Hereby the pressure required for the back support is pointed partly forwards and partly downwards. This may be provided by the straps according to embodiments of the present invention.

A major advantage of the present invention is the comfortability. As mentioned, the prior art back support is generally unpleasant to wear, which has the disadvantage that people tend to only wear it for limited time. Instead a back support according to embodiments of the present invention allows for prolonged use of a back support without discomfort. In preferred embodiments, the inventive back support may be worn daily/all day whereby the risk of recurrence in connection with back injuries may be minimized.

The back support according to the invention may be used anywhere, such as in hospitals, care homes, and private homes. It may be used with back damages and back problems as well as in adjusting posture or merely for comfort reasons.

1. A back support comprising:
a waist band with at least one front-facing fixing region and at least one back-facing supporting plate attached to said waist band; and
at least two first leg straps fixed in both ends unto said waist band in said fixing region;
said first leg straps each forming a loop extending in use from the fixing region downwardly and behind a respective leg of the user and again upwardly to the fixing region;
said at least one supporting plate being adapted to support the lower back region of the user.

2. A back support according to claim 1, wherein said back support further comprises at least two second leg straps fixed in both ends unto said waist band in said fixing region; said second leg straps each forming a loop extending in use from the fixing region downwardly and behind a respective leg of the user and again upwardly to the fixing region; wherein said at least two first leg straps are adapted for, when in use, tightening behind the upper region of the thigh of the user, and said at least two second leg straps are adapted for, when in use, tightening behind the lower region of the thigh of the user.

3. A back support according to claim 1, wherein said supporting plate is substantially shaped as the shape of a back of a user to increase the comfortability for the user and/or said supporting plate comprises a pad to increase the comfortability for the user.

4. A back support according to claim 1, wherein said back support further comprises a connecting means for connecting said waist band to a fixed structure in front of the user, preferably said connecting means comprises a hook, a snap hook and/or a strap comprising a loop.

5. A back support according to claim 1, wherein said back support further comprises a lower strap system for, when in use, assisting said second leg straps in maintaining their position on the lower region of the thigh of the user; said lower strap system comprising at least one strap surrounding the leg of said user;
said lower strap system, when in use, comprises two straps on each leg of said user, one above the knee and one below the knee, both surrounding the legs, and at least one connecting strap for connecting said straps on each leg.

6. A back support according to claim 1, wherein the straps are formed of an elastic material.

7. A back support according to claim 1, wherein said at least one back-facing supporting plate has an area of more than 100 cm².

8. A back support according to claim 1, wherein said at least one back-facing supporting plate is fixed on said waist band or said waist band is connected to said supporting plate in each side of said supporting plate.

9. A back support according to claim 1 configured for supporting the back of a user.

10. Method of supporting the back of a user by wearing a back support according to claim 1.
